(19) United States
(12) Patent Application Publication
(10) Pub. No.: US 2025/0235661 A1
(43) Pub. Date: Jul. 24, 2025

(54) System And Method For Regulating Biobehavioral States

(71) Applicant: Polyvagal Music LLC, Atlantic Beach, FL (US)

(72) Inventors: Stephen W. Porges, Atlantic Beach, FL (US); Anthony Gorry, Suffolk (GB)

(73) Assignee: Polyvagal Music LLC, Atlantic Beach, FL (US)

(21) Appl. No.: 18/846,500

(22) PCT Filed: Sep. 4, 2024
(86) PCT No.: PCT/US2024/045129

§ 371 Date: Sep. 12, 2024

Related U.S. Application Data
(60) Provisional application No. 63/539,435, filed on Sep. 20, 2023.

(51) Int. Cl. A61M 21/02; A61M 21/00; G16H 20/70

(52) U.S. Cl. CPC A61M 21/02; G16H 20/70; A61M 2021/0027; A61M 2205/3303; A61M 2205/3327; A61M 2230/005; A61M 2230/04; A61M 2230/42

(57) ABSTRACT

A system and method for composing and processing music to regulate autonomic and related physiological systems (e.g., endocrine, immune) and biobehavioral states is disclosed. The system can include a music composition module, a music delivery module, and a feedback module attached to a listener to allow for the real-time regulation of autonomic and related physiological systems and biobehavioral states. The music delivery module can modulate one or more audio signals being transmitted to the user to assist with the regulation of the user's autonomic and related physiological systems and biobehavioral state.

### CLAIM OF PRIORITY

This application claims priority to U.S. Provisional Patent Application No. 63/539,43, filed on Sep. 20, 2023, entitled “System and Method for Regulating Biobehavioral States.”

### NOTICE OF COPYRIGHTS AND TRADE DRESS

A portion of the disclosure of this patent document contains material which is subject to copyright or trade dress protection. This patent document may show and/or describe matter that is or may become trade dress of the owner. The copyright and trade dress owner has no objection to the facsimile reproduction by anyone of the patent disclosure, as it appears in the Patent and Trademark Office patent files or records, but otherwise reserves all copyright and trade dress rights whatsoever.

### FIELD OF THE EMBODIMENTS

The invention in accordance with the present disclosure relates generally to the field of regulating biobehavioral and autonomic states including, but not limited to, convergent influences on endocrine and immune systems, and more specifically to modulating acoustic parameters within musical compositions utilizing endogenous bodily rhythms, and utilizing the modulated musical compositions with the embedded bodily rhythms to regulate biobehavioral and autonomic states and related physiological systems (e.g., immune, endocrine, histamine) by delivering such modulated acoustic and/or vibroacoustic stimulation. Methods of reflecting endogenous bodily rhythms via patterns of acoustic and/or vibroacoustic stimulation, music and/or composed processed music via acoustic and vibroacoustic stimulation are engineered to regulate biobehavioral state and related physiological systems including the neural regulation of visceral organs via the autonomic nervous system are also provided.

### BACKGROUND

The potential therapeutic benefits of music and acoustic and/or vibroacoustic stimulation to enhance the regulation of the nervous system to influence function of autonomic and related physiological systems, behavior, and biobehavioral states have been recognized. However, existing approaches are inadequate as they lack precision in both delivering and assessment of the actual rhythms through which the brain is communicating with the bodily organs associated with the autonomic nervous system and related physiological systems. Further, even for such systems that do attempt to regulate behavior, autonomic function and related physiological systems, and biobehavioral states, these systems lack the ability to be tuned to the endogenous physiological rhythms expressed by the autonomic nervous system and related physiological systems, to be meaningfully customized to the user's endogenous physiological rhythms, and to provide any real-time feedback for or about the user's biobehavioral state including the endogenous rhythms expressed by the user's autonomic nervous system and other related physiological systems. Especially, these systems lack the ability to use music and other forms of acoustic and vibroacoustic stimulation to entrain the slower endogenous rhythms regulating homeostatic functions of visceral organs that may involve the vagus and especially the branch of the vagus originating in a dorsal area of the brainstem known as the dorsal motor nucleus of the vagus (i.e., dorsal vagus).

Therefore, there exists a need for a device that combines in the composition of music, endogenous bodily rhythms and acoustic properties of sound waves and other audio and vibroacoustic transmissions to beneficially regulate the biobehavioral state dependent on the neural regulation of a listener's autonomic nervous system and related physiological systems.

Specifically, there is a need for a system and method that can align rhythmic structure of acoustic and/or vibroacoustic features within the music composition including tempo, bandwidth, volume, and dispersion with the listener's endogenous bodily rhythms, which would signal the nervous system, to promote entrainment and enhance the desired autonomic (including related physiological systems) and biobehavioral states. Finally, there exists a need for a device that can facilitate the entrainment of bodily rhythms with music and other acoustic and/or vibroacoustic simulation that has embedded features that incorporate the rhythms expressed in the listener's heart rate, respiratory rate, gut peristalsis or other bodily processes such as rhythms reflected in the vascular system, oxygenation in the blood, uterus, endocrine system, immune system, and cerebral fluid. The present disclosure meets and exceeds these objectives.

### SUMMARY OF THE INVENTION

The system and method in accordance with the present disclosure have a wide range of applications in fields such as, but not limited to, music therapy, relaxation techniques, stress management, mood enhancement, sleep, sports or other forms of physical and mental performance, education, psychotherapy, psychiatry, internal medicine, functional medicine, psychosomatic medicine, anesthesiology, pain medicine, chronic illness, recovery from illness and injury, rehabilitation medicine, performance, and overall health and well-being. The personalized and synchronized nature of the music results in effectively regulating the autonomic nervous system including related physiological systems and biobehavioral states by catering to individual features of neural regulation, preferences, and needs. In addition, the technology can be modified to support the autonomic nervous system and other related physiological systems and biobehavioral states of other mammals to calm pets and to potentially increase the health and wellbeing of domesticated animals. For example, applications could include enhancing growth trajectories (e.g., weight gain) and milk production in domesticated livestock and calming pets, especially those that have been rescued from adverse environments. Functionally, the goal is to enable the listener's autonomic nervous system to calm and ‘immobilize without fear,” a state that promotes the homeostatic functions of health, growth, and restoration. Based on Polyvagal Theory, this state hypothetically requires optimizing the neural regulation of visceral organs via vagal pathways originating in the brainstem in an area known as the dorsal nucleus of the vagus.

The system and method in accordance with the present disclosure are configured to regulate autonomic and related physiological systems and biobehavioral state through the modulation of tempo, frequency bandwidth, sound dispersion, and other acoustic and vibroacoustic properties via endogenous bodily rhythms either estimated from literature (normative data based on species, physical size, and age) or directly monitored for the individual. This functionality offers significant advancements in several fields concerned with optimizing the experience and performance of humans and animals. By leveraging more accurate and precise dynamic adjustments, the invention provides a powerful tool for enhancing physiological and psychological well-being including supporting health and restoration by optimizing homeostatic function of physiological systems.

The present disclosure provides for a system for regulating autonomic nervous system and related physiological systems and biobehavioral states. In some embodiments this system includes a music composition module and a music delivery module, and in other embodiments this system also includes a feedback module. In some embodiments, the music composition module will be configured to modulate uniquely composed music that has embedded rhythmicities associated with one or more endogenous bodily rhythms, the music delivery module will have at least one sound transmittal mechanism configured to deliver the modulated musical composition. The feedback module will be configured to monitor one or more physiological properties of a listener via one or more sensors. In some embodiments, the sound transmittal mechanism is configured to deliver music composed and processed by an attached music composition module in accordance with the present disclosure. For the purposes of this disclosure, music or music composition can be interpreted to mean one or more sounds frequencies within the spectra of either or both acoustic and vibroacoustic sounds.

In at least one embodiment, the music composition module is configured to modulate certain properties of the music composition including the modulation of tempo, volume, spectral energy distribution, laterality, dispersion, melodic patterns, and one or more bandwidth frequencies. In some embodiments, one of the bandwidth frequencies shall have a predetermined upper bound and having a predetermined lower bound which are informed by the species (and age) specific resonance frequency and transfer function of middle ear structures. The modulation of acoustic and vibroacoustic parameters at different periodicities are engineered to be harmonically related. This enables the different modulated frequencies to synchronize through resonance and constructive interference, leading to a coherent and harmonious interaction among the different frequencies. Embodiments exist where the music composition module is configured to embed endogenous biological rhythms in the vibroacoustic and/or acoustic parameters of the musical composition. In various embodiments, the sound transition mechanism utilizes digital streaming platforms; analog physical media, digital physical media, and/or high-fidelity sound transmission devices. In other embodiments the sound transition mechanism may be integrated into a virtual or augmented reality or gaming platform. In other embodiments, the music delivery module may be integrated with a technology that would provide an immersive sound experience via 360-degree surround sound and other current and future immersive sound technologies. In other embodiments, the music delivery module may be configured to dynamically modulate sound dispersion and may be combined with modulation of tempo and bandwidth or other acoustic parameters (e.g., volume, laterality) at endogenous biological rhythms.

In various embodiments, the feedback module includes at least one physiological and/or behavioral sensor configured to interface with the listener, and at least one behavioral and/or physiological sensor can be a device which measures heart rate or pulse rate and rhythm, respiratory rate and rhythm, peristaltic rhythms of the gut, peristaltic rhythm of the uterus, vasomotor rhythm, blood pressure rhythm, oxygenation rhythm, movement, muscle tension, blood concentrations of neurochemicals such as hormones or cytokines, and/or cerebral spinal fluid rhythm. In an embodiment, the music composition module is configured to manipulate music being transmitted by the music delivery module based on the endogenous rhythms of one or more physiological properties monitored by the feedback module. For example, a behavioral sensor could be a sensor which assessed movement via the utilization of an accelerometer.

The present disclosure also contemplates a method for regulating autonomic and related physiological systems and biobehavioral states. In some embodiments, the method begins by configuring at least one behavioral and/or physiological sensor such that the at least one behavioral and/or physiological sensor monitors one or more physiological properties of a listener. The method may then proceed to the step of connecting the at least one behavioral and/or physiological sensor to a feedback module. From there the method may proceed to the step of providing a music composition module, configured to embed the endogenous rhythms in the acoustic and/or vibroacoustic features of the music. From there, the method may proceed to the step of transmitting by the music delivery module, music, to the listener and then monitoring by the at least one behavioral or physiological sensor one or more behavioral and/or physiological properties. Based on the physiological properties that are monitored, the music composition module can then manipulate the music by modulating the acoustic and/or vibroacoustic features at a rhythmicity that more closely matches the user's endogenous physiological rhythms. Then these manipulated sound frequencies are transmitted to the listener by the music delivery module. The steps of monitoring, manipulating, and transmitting may be repeated as needed or desired. In some embodiments the composed music may also be manipulated by having the sound dispersion being selectively modified.

The present disclosure also provides for a method of modulating a musical composition. In various embodiments of this method, the first step is to provide a musical composition to be modulated. The method can then proceed to breaking down the musical composition into one or more subcomponents. A normative endogenous bodily rhythm is then provided, and the one or more subcomponents are manipulated to partially or completely confirm to the normative endogenous bodily rhythm. The manipulated one or more subcomponents is then substituted for the original one or more subcomponents in the musical composition, resulting in a modulated musical composition. In some embodiments, this method can proceed to the step of providing at least one biobehavioral or physiological sensor and then attaching the sensor to the listener. Once attached, the sensor will monitor one or more biobehavioral or physiological properties of the listener. Then, the one or more subcomponents of the musical composition are further manipulated by comparing the normative endogenous bodily rhythm to the monitored biobehavioral or physiological properties in the previous step. The modified composition is then transmitted to the listener, and these steps can all be repeated as desired.

The claims should not necessarily be construed as limited to addressing any of the particular problems or deficiencies discussed hereinabove. To the accomplishment of the above, this disclosure may be embodied in the form illustrated in the accompanying drawings. Attention is called to the fact, however, that the drawings are illustrative only. Variations are contemplated as being part of the disclosure.

Implementations may include one or a combination of any two or more of the aforementioned features or embodiments.

These and other aspects, features, implementations, and advantages can be expressed as methods, apparatuses, systems, components, program products, business methods, and means or steps for performing functions, or some combination thereof.

Other features, aspects, implementations, and advantages will become apparent from the descriptions, the drawings, and the claims.

### BRIEF DESCRIPTION OF THE DRAWINGS

In the drawings, like elements are depicted by like reference numerals. The drawings are briefly described as follows.

FIG. 1 is a flow chart depicting the steps of an embodiment of the method of regulating autonomic nervous system and related physiological systems, and biobehavioral states in accordance with the present disclosure.

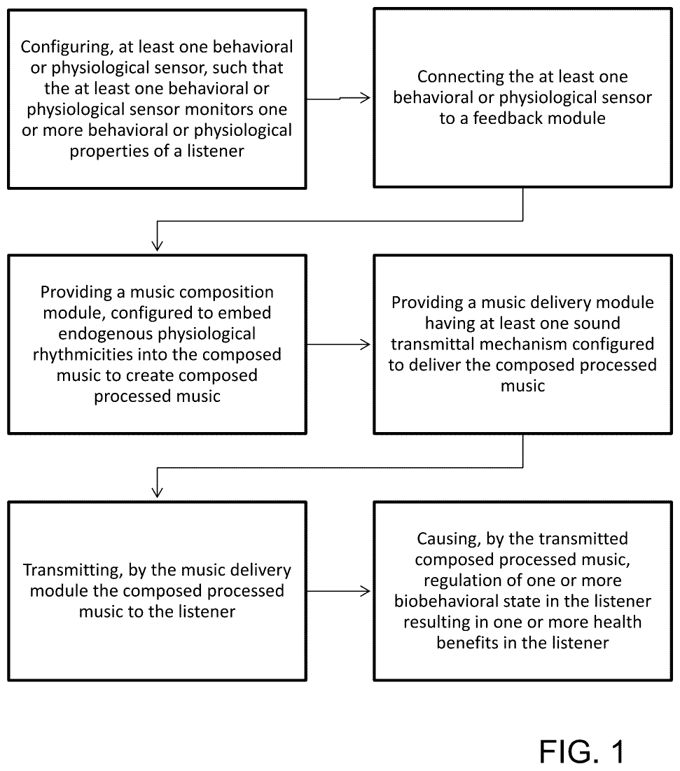

FIG. 2 is a flow chart depicting the steps of an embodiment of the method of modulating a musical composition in accordance with the present disclosure.

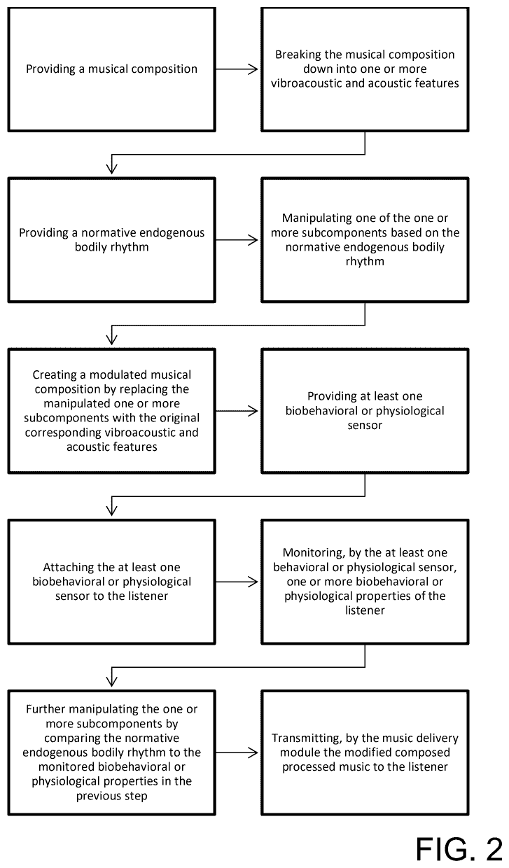

FIG. 3 is a chart illustrating embedded endogenous rhythms in the beat-to-beat heart rate patterns, highlighting the endogenous rhythms associated with respiration, blood pressure, and vasomotor tone.

FIG. 4 is a chart illustrating normal vs abnormal gut peristalsis rhythms.

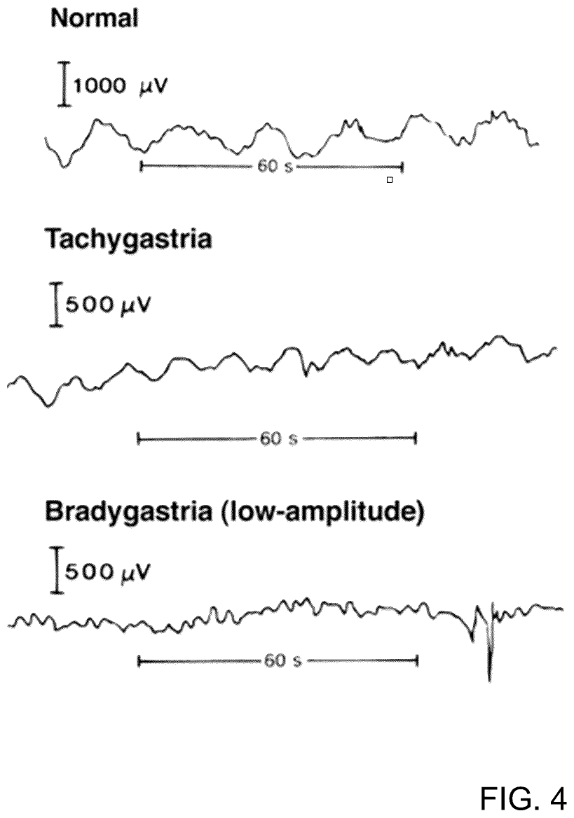

The present disclosure now will be described more fully hereinafter with reference to the accompanying drawings, which show various example embodiments. However, the present disclosure may be embodied in many different forms and should not be construed as limited to the example embodiments set forth herein. Rather, these example embodiments are provided so that the present disclosure is thorough, complete, and fully conveys the scope of the present disclosure to those skilled in the art. In fact, it will be apparent to those skilled in the art that various modifications and variations can be made in the present invention without departing from the scope or spirit of the invention.

### DETAILED DESCRIPTION

Reference will now be made in detail to each embodiment of the present invention. Such embodiments are provided by way of explanation of the present invention, which is not intended to be limited thereto in any manner whatsoever. In fact, those of ordinary skill in the art may appreciate upon reading the present specification and viewing the present drawings that various modifications and variations can be made thereto.

For purposes of the present disclosure of the invention, unless specifically disclaimed, the singular includes the plural and vice-versa, the words “and” and “or” shall be both conjunctive and disjunctive, the words “any” and “all” shall both mean “any and all”.

Functionally, the invention in accordance with the present disclosure signals the nervous system via entrainment by acoustic and vibroacoustic stimulation to regulate autonomic state and related physiological systems to influence biobehavioral states including states that would promote alertness, relaxation, and health. The present disclosure provides for a system comprising a music composition module, a music delivery module, and an optional feedback module. In various embodiments, the music composition module is to be used by a composer who is modulating sound or otherwise augmenting the sound of a musical composition. In some embodiments, the music composition module will directly apply one or more algorithms to linearly and rhythmically modulate the tempo, frequency bandwidth, sound dispersion parameters, volume, spectral energy distribution, laterality, and other acoustic and/or vibroacoustic properties of the music being transmitted to at least one listener. In other embodiments, the music composition module is utilized by a human user directly modifying a given musical composition. Like the above, in these embodiments the user will be able to linearly and rhythmically modulate the tempo, frequency, bandwidth, sound dispersion parameters, volume, spectral energy distribution, laterality, and other acoustic and/or vibroacoustic properties of said musical composition.

In some embodiments, the algorithm will be based on normative endogenous bodily rhythms of the listener or an estimate based on published normative data based on age, size, and species. In some embodiments the algorithm and related techniques are incorporated to ensure seamless transitions and optimal synchronization with the specific listener's endogenous bodily rhythms. In some embodiments, the system and method in accordance with the present disclosure can be used to exercise or train the listener's nervous system to build resilience to enhance spontaneous recovery of autonomic state and related physiological systems following physical, medical, emotional, cognitive, or psychosocial challenges. In some forms, this embodiment may function as an adjunctive therapy supporting other clinical treatments.

The music delivery module may utilize various media for transmitting the music to the listener or listeners. Such media may include digital streaming platforms, physical media, or specialized devices capable of reproducing the music with fidelity and accuracy.

In some embodiments, the music delivery module may interface with a feedback module. This feedback module incorporates physiological sensors or an interface to capture real-time bodily data, such as heart rate, gastrointestinal motility, respiratory rate, and/or other relevant physiological signals to identify the listeners endogenous bodily rhythm or rhythms. In some embodiments, the feedback module will employ one or more algorithms and processing techniques to analyze the captured bodily data and dynamically adjust the musical elements during playback. This real-time modulation can ensure optimal alignment with the listener's physiological and psychological states, facilitating the regulation of biobehavioral responses and supporting health via optimizing homeostatic processes. By utilizing knowledge of the endogenous rhythms reflecting homeostatic feedback mechanisms in the autonomic nervous system and related physiological systems, music transmitted by the music delivery module provides for a unique and efficient neuroacoustic intervention with the listener. Uses can include reduction of stress, facilitation of homeostatic functions, enhanced sociality, improved learning, and overall optimized health and performance.

Various endogenous bodily rhythms can be modulated within the acoustic and vibroacoustic stimulation to regulate biobehavioral states of the listener as the state of the autonomic nervous system and related physiological systems are spontaneously and dynamically expressed in several easily monitored endogenous rhythms of the body. For example, the frequencies expressed in heart rate, respiration, and vascular tone are not only easily monitored, but can be felt. Ballistographic recordings confirm that these rhythmic autonomic processes literally cause movement within the body at identifiable frequencies.

One such endogenous bodily rhythm that can be modulated is the heart rate rhythm. This rhythm is the prominent rhythm observed in the vascular system and refers to the regular pattern of heartbeats. These heartbeats are controlled by electrical signals generated by the heart's natural pacemaker, the sinoatrial (SA) node. In some embodiments, the heart rate rhythm is modulated between 1-1.7 Hz, or 60-100 beats per minute and can be monitored via an electrocardiogram (ECG) or a pulse rhythm via a photoplethysmography. The heart rate rhythm is age, size, and species dependent. The values listed for endogenous rhythms represent normative data from healthy adults.

Another such endogenous bodily rhythm is the respiratory rhythm. The respiratory rhythm refers to the pattern of breathing, specifically the inhalation and exhalation of air. It is controlled by the respiratory center in the brainstem. During inhalation, oxygen is taken in, and during exhalation, carbon dioxide is expelled. This rhythmic process ensures a constant supply of oxygen and removal of carbon dioxide from the body. The pattern of breathing influences the calming influence of the vagus on the heart with profound slowing of heart rate during exhalation. In some embodiments, in healthy adults the respiratory rhythm is modulated between 0.12-0.40 Hz or 7.2-24 breaths per minute.

Other such endogenous bodily rhythms that the system and method in accordance with the present disclosure can utilize are the vasomotor and baroreflex rhythms. Vasomotor rhythms are rhythmic fluctuations in vascular tone that can occur at frequencies slower than spontaneous breathing. These rhythms reflect the dynamic regulation of blood vessel constriction and dilation. Blood pressure regulation through the baroreflex is an assumed mechanism, since baroreceptor reflexes typically operate in the low-frequency range of around 0.05 Hz to 0.1 Hz or 3 to 6 per minute and can be observed as fluctuations in blood pressure.

Blood pressure and vasomotor rhythms can occur at similar frequencies. However, while vascular rhythms, vasomotor rhythms, and blood pressure rhythms share common mechanisms, they represent distinct aspects of cardiovascular function. Vascular rhythms can encompass broader phenomena related to blood vessels. Vasomotor rhythms specifically refer to variations in vascular tone and blood pressure rhythms pertain to rhythmic fluctuations in arterial pressure. The interconnectedness of these rhythms reflects the complex regulation of cardiovascular dynamics that co-occur and are entrained at a common endogenous frequency. As the vasomotor and baroreflex rhythms can occur at similar frequencies, both of these rhythms can be modulated between 0.05 to 0.1 Hz or 3 to 6 per minute. In preliminary tests of entraining listeners with normative endogenous blood pressure and vasomotor rhythms, listeners engaged in 10-30 minutes sessions of listening to the modulated musical compositions as an adjunctive therapy to other forms of therapy. Of the 7 participants, 6 had improved scores on the Nijmegen Questionnaire (NQ), and three of those participants transitioned from a positive indication of hyperventilation syndrome to normal.

Yet another endogenous bodily rhythm that can be modulated is the cerebral spinal fluid rhythm. Cerebrospinal fluid dynamics involve several rhythms, similar in frequency to a standard heart rate, respiration, as well as the slower vasomotor and blood pressure rhythms. The rhythms observed in cerebrospinal fluid dynamics are crucial for maintaining brain homeostasis, waste clearance, and normal brain function. It is noted that there are other neural rhythms, such as brain (electroencephalograph) and gut (electrogastrograph) rhythms that may be used by the system and method in accordance with the present disclosure to modulate acoustic properties embedded in the musical compositions. In other preliminary tests, out of a population of 8 test listeners, 7 showed improvement on the Brain-Body Center Sensory Scales, with the largest improvements being observed in digestion, auditory hypersensitivity, and affiliative touch aversion domains. Additionally, 7 of these 8 listeners showed a reduction of autonomic reactivity, which is associated with autonomic dysregulation, which is associated with chronic stress. Moreover, on the Neuroception of Psychological Safety Scale, 6 of 7 clients improved on the social engagement subscale reflecting a greater ease in their ability to socially interact.

In various embodiments, variations in the phase relationship between modulated acoustic parameters can exist. For example, it is possible to modulate volume and bandwidth with a 180 degree phase difference, resulting in volume increases while bandwidth decreases in the composed processed music.

Generally, when a mammal experiences metabolic demands, perceives threat, and/or experiences illness, the amplitude and prevalence of these endogenous bodily rhythms is reduced. Functionally, these rhythms represent the negative feedback circuits that support homeostatic functions, and physiologically, this can be confirmed by monitoring aspects of the autonomic nervous system.

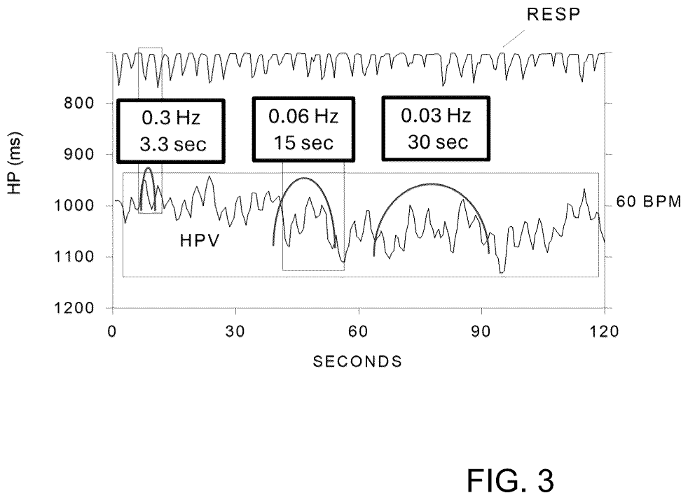

Referring to FIG. 3, a chart illustrating embedded endogenous rhythms within a beat-to-beat heart rate pattern is provided. Within this chart are high-frequency oscillations representing respiratory sinus arrhythmia. These high-frequency oscillations in heart rate variability occur in the range of approximately 0.12 to 0.4 Hz (or 8.3 to 2.5 sec a cycle or 7.2 to 24 cycles per minute). These oscillations primarily reflect respiratory sinus arrhythmia, the variation in heart rate synchronized with the respiratory cycle and are mediated by ventral vagal pathways. During inspiration, vagal influences are dampened and heart rate increases, and during expiration, vagal influences recover and heart rate decreases. Low-frequency oscillations are also shown, and within heart rate variability occur in the range of approximately 0.04 to 0.12 Hz (or 2.4 to 8 cycles per minute). The physiological mechanisms underlying low-frequency oscillations are more complex and may involve sympathetic and parasympathetic influences, baroreflex activity, and other autonomic nervous system components. Additionally, very low-frequency oscillations are also shown and within heart rate variability occur at frequencies below 0.04 Hz (or less than 2.4 cycles per minute). These rhythms are assumed to be related to other regulatory processes such as thermoregulation, hormonal fluctuations, and circadian rhythms. In an embodiment, the method in accordance with the present disclosure involves receiving input from the composer regarding desired musical elements, as well as endogenous bodily rhythms to target. The optional feedback module enables collaboration between the composition and delivery modules to create personalized music that dynamically modulates tempo, frequency bandwidth, volume, spectral energy distribution, laterality, sound dispersion, and other acoustic properties based on the individual's current profile of bodily rhythms, as can be recorded in real-time by the feedback module in accordance with the present disclosure.

Musical compositions, as transmitted by the music delivery module should not be composed with ‘fixed’ or constant acoustic properties. Various parameters can be dynamically changed through the composition, in some embodiments by an algorithm that modulates the feature in a temporal manner that is, informed by knowledge of the frequency characteristics of endogenous bodily rhythms.

One such property of such a musical composition is the tempo, as adjusting the tempo of a musical composition can have an impact on the listener's physiological and biobehavioral state. For example, increasing the tempo through linear contraction can promote alertness and energize the listener, while decreasing the tempo through linear expansion can induce relaxation and calmness. In various embodiments, such musical compositions will be composed with a pattern of tempo variation in which tempo, informed by endogenous bodily rhythms, is rhythmically modulated. Some embodiments will embed the listener's heart rate and respiration frequencies, although other embodiments exist where slower vasomotor/blood pressure or peristaltic rhythms of the gut are incorporated. In some embodiments, three features of tempo modulation will be incorporated into the composition: tempo frequency, duration of tempo modulation sequences, and range of tempo changes within said modulation sequence.

In accordance with the present disclosure, tempo for each composition will be centered at a frequency associated with a biobehavioral state such that slower frequencies reflecting slower heart rate will be applied to entrain the listener's physiological state to calm, relax, and perhaps foster sleep, while faster frequencies will be applied to increase arousal for energetic and directed movement and tasks. In some embodiments, the tempo will be changing systematically on each beat of the composition creating a systematic sequence over defined period of a linear expansion or contraction of tempo. In other embodiments, the tempo will be changing systematically on each beat of the composition creating a systematic predictive sequences of tempo increases followed by a symmetrical sequence of tempo decreases. For example, the duration of the sequence could be determined by the characteristic breathing frequency associated with the preferred biobehavioral state. Alternatively, tempo could be modulated at the slower vasomotor/blood pressure rhythm or both the respiratory and vasomotor/blood pressure rhythms could be embedded. Finally, the range of tempo changes refers to the degree that tempo will change within said tempo duration cycle. For example, a moderate sequence of tempo change may optimize entrainment and calming while large change may be arousing, and a small change may be ineffective. As another example, tempo may be modulated by expanding the tempo in a linear fashion, which is assumed to have a benefit in assisting listeners in getting to sleep.

It is noted that modulating the frequency bandwidth within a music composition requires manipulating the range of frequencies present in the acoustic spectrum. This can be achieved through equalization and filtering techniques that produce a rhythmic pattern of increasing and decreasing bandwidth within defined upper and lower frequencies. The rhythmic pattern will be informed by knowledge of endogenous bodily rhythms, which may be derived offline or in real-time from the feedback module in accordance with the present disclosure.

It is also noted that different frequency ranges can evoke specific emotional responses and influence the listener's state of mind. Research documents that there is a frequency band of perceptual advantage that can be defined for each mammalian species (see Porges & Lewis 2010, Kolocz, Lewis, & Porges, 2018). Such a frequency band is used for social communication and frequently used to signal safety or lack thereof.

Polyvagal theory explains that the ability to process acoustic information within this frequency band is dependent on the transfer function of middle ear structures, which is mediated by physiological state. Functionally, a calm physiological state promoted by the ventral vagal complex is paralleled by increases in the activation of middle ear muscles tensing the ossicle chain and the eardrum. These changes can result in low frequency background sounds literally bouncing off the eardrum and thereby optimizing auditory processing by facilitating the extraction of speech and other acoustic forms of social communication. Contraction of the middle ear muscles function as a filter increasing the signal to noise ratio of the acoustic properties within this frequency band. Thus, the frequency band of perceptual advantage is dependent on the physics of the middle ear structures as well as the state of contraction of the middle ear muscles.

The music delivery module in accordance with the present disclosure may be configured to manipulate sound dispersion, as well as to utilize spatial effects (e.g., 360-degree surround sound system) which can enhance the immersive and therapeutic qualities of music. For example, the system and method in accordance with the present disclosure proposes a novel application in which music will be composed by embedding a pattern of sound dispersion variations, in which the degree of sound dispersion systematically increases and decreases over a duration informed by the selected endogenous bodily rhythm. Additionally, other acoustic properties, such as reverberation, timbre, and dynamics, may also be embedded by being modulated by one or more endogenous bodily rhythms. In addition, the selection and arrangement of instruments, including their tonal qualities, relative volume, spatial placement, and harmonic interactions, may also be rhythmically embedded, as these properties are capable of enhancing the intended physiological and psychological responses in the listener.

It is understood that when an element is referred hereinabove as being “on” another element, it can be directly on the other element or intervening elements may be present therebetween. In contrast, when an element is referred to as being “directly on” another element, there are no intervening elements present.

Moreover, any components or materials can be formed from a same, structurally continuous piece or separately fabricated and connected.

Features illustrated or described as part of one embodiment can be used with another embodiment and such variations come within the scope of the appended claims and their equivalents. Implementations may also include one or a combination of any two or more of the aforementioned features or embodiments.

Example embodiments described herein should not be construed as limited to the particular shapes of regions as illustrated herein, but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

As the invention has been described in connection with what is presently considered to be the most practical and various embodiments, it is to be understood that the invention is not to be limited to the disclosed embodiments, but on the contrary, is intended to cover various modifications and equivalent arrangements included within the scope of the appended claims. Although specific terms are employed herein, they are used in a generic and descriptive sense only and not for purposes of limitation.

This written description uses examples to disclose the invention, including the best mode, and also to enable any person skilled in the art to practice the invention, including making and using any devices or systems and performing any incorporated methods. The patentable scope of the invention is defined in the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

The claims should not necessarily be construed as limited to addressing any of the particular problems or deficiencies discussed hereinabove. To the accomplishment of the above, this disclosure may be embodied in the form illustrated in the accompanying drawings. Attention is called to the fact, however, that the drawings are illustrative only. Variations are contemplated as being part of the disclosure.

The disclosure is illustrated throughout the written description. It should be understood that numerous variations are possible while adhering to the inventive concept. Such variations are contemplated as being a part of the present disclosure.

What is claimed is:

1. A system for regulating autonomic and related physiological systems and biobehavioral states, comprising:
a music composition module, configured to rhythmically modulate vibroacoustic and acoustic features of a musical composition,
wherein the modulations are performed at periodicities defined by endogenous biological rhythms,
wherein the music composition module is configured to embed endogenous biological rhythms in the vibroacoustic and acoustic parameters of the musical composition;
a music delivery module having at least one sound transmittal mechanism configured to deliver the modulated musical composition,
wherein the music composition module and the music delivery module are in communication.

2. The system of claim 1, further comprising a feedback module configured to monitor one or more behavioral or physiological properties of a listener,
wherein the feedback module is in communication with the music composition module and the music delivery module,
wherein the feedback module is configured to monitor one or more biobehavioral or physiological properties of a listener and configured to transmit the one or more biobehavioral or physiological properties to the music composition module,
wherein the embedded endogenous biological rhythms are being informed by one or more physiological or behavioral properties monitored by the feedback module.

3. The system of claim 2, the music composition module being configured to rhythmically manipulate the properties of the one or more acoustic and/or vibroacoustic parameters, the parameters selected from the group comprising rhythmic structure, tempo, melodic patterns, bandwidth frequencies, volume, spectral energy distribution, sound dispersion, and laterality.

4. The system of claim 3, the bandwidth frequency having a predetermined upper bound and having a predetermined lower bound.

5. The system of claim 4, wherein the predetermined upper bound and the predetermined lower bound are rhythmically modulated through use of an endogenous bodily rhythm.

6. The system of claim 5, wherein the at least one sound transition mechanism is selected from the group consisting essentially of: digital streaming platforms; analog physical media, digital physical media, and high-fidelity sound transmission devices.

7. The system of claim 5, wherein the music delivery module is configured to dynamically modulate sound dispersion of the delivered modulated musical composition based on input from the music composition module.

8. The system of claim 7, wherein the music delivery module is configured to dynamically modulate sound dispersion of the delivered modulated musical composition also based on input from the feedback module.

9. The system of claim 1, the feedback module further comprising at least one physiological sensor configured to interface with a listener.

10. The system of claim 7, the one or more physiological properties being selected from the group comprising heart rate or pulse rhythm, respiratory rhythm, vasomotor rhythm, blood pressure rhythm, peristaltic rhythm of the gut, cerebral spinal fluid rhythm or other rhythm intrinsic to the body and observed in a visceral organ, bodily fluid, or vascular tissue.

11. A method of regulating biobehavioral states, comprising the steps of:
a. configuring, at least one behavioral or physiological sensor, such that the at least one behavioral or physiological sensor monitors one or more behavioral or physiological properties of a listener;
b. connecting the at least one behavioral or physiological sensor to a feedback module;
c. providing a music composition module, configured to embed endogenous physiological rhythmicities into the composed music to create composed processed music;
d. providing a music delivery module having at least one sound transmittal mechanism configured to deliver the composed processed music;
e. transmitting, by the music delivery module the composed processed music to the listener;
f. causing, by the transmitted composed processed music, regulation of one or more biobehavioral state in the listener resulting in one or more health benefits in the listener.

12. The method of claim 11, the one or more health benefits being selected from the group comprising facilitating sleep, enhancing recovery from exercise or injury, supporting homeostatic processes and health including enhancing endocrine and immune function, enhancing relaxation, reducing physiological and psychological features of stress, and promoting enhanced mental and physical performance.

13. The method of claim 12, the music composition module being further configured to modulate tempo of the composed music via linear expansion and contraction.

14. A method of modulating a musical composition, comprising the steps of:
a. providing a musical composition;
b. breaking the musical composition down into one or more vibroacoustic and acoustic features;
c. providing a normative endogenous bodily rhythm;
d. manipulating one of the one or more subcomponents based on the normative endogenous bodily rhythm; and
e. creating a modulated musical composition by replacing the manipulated one or more subcomponents with the original corresponding vibroacoustic and acoustic features.

15. The method of claim 14, wherein the one or more subcomponents is selected from the group comprising rhythmic structure, tempo, melodic patterns, bandwidth frequencies, volume, spectral energy distribution, sound dispersion, and laterality.

16. The method of claim 14, the normative endogenous bodily rhythm being selected from the group comprising heart rate or pulse rhythm, respiratory rhythm, vasomotor rhythm, blood pressure rhythm, gut peristalsis, uterine peristalsis, cerebral spinal fluid rhythm or other rhythm intrinsic to the body and observed in a visceral organ, bodily fluid, or vascular tissue.

17. The method of claim 14, further comprising the steps of:
f. Providing at least one biobehavioral or physiological sensor;
g. attaching the at least one biobehavioral or physiological sensor to the listener,
h. monitoring, by the at least one behavioral or physiological sensor, one or more biobehavioral or physiological properties of the listener,
i. further manipulating the one or more subcomponents by comparing the normative endogenous bodily rhythm to the monitored biobehavioral or physiological properties in the previous step;
j. transmitting, by the music delivery module the modified composed processed music to the listener; and
k. repeating steps f-j, as desired.

18. The method of claim 17, wherein the composed processed music is manipulated by modulating a range of frequencies present in the acoustic and/or vibroacoustic spectra of the music composition.

19. The method of claim 17, wherein the modified musical composition is configured as a neural exercise to enhance homeostatic function to support regulation of visceral organs of the listener.
